(19) United States
(12) Patent Application Publication
(10) Pub. No.: US 2007/0248656 A1
(43) Pub. Date: Oct. 25, 2007

(54) TOPICAL PREPARATION DISPENSERS AND METHODS OF USING THE SAME

(76) Inventor: Bradley S. Galer, West Chester, PA (US)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 11/692,057

(22) Filed: Mar. 27, 2007

Related U.S. Application Data
(60) Provisional application No. 60/745,570, filed on Apr. 25, 2006.

(51) Int. Cl. A61F 13/02; A61K 31/65; A61K 31/555; A61K 31/496; A61K 31/7034; A61K 31/7048; G07F 11/00

(52) U.S. Cl. 424/448; 221/69; 424/618; 514/28; 514/152; 514/184; 514/35; 514/252.13

(57) ABSTRACT

Topical preparation dispensers and methods of using the same are provided. Aspects of the invention include a topical preparation dispenser having a holder, one or more vertically stacked topical preparations in the holder, and a cap at one end of the holder configured such that upon movement of the cap, a topical preparation is exposed and moved relative to the holder. Also provided are kits that include the subject dispensers.

### CROSS-REFERENCE To RELATED APPLICATIONS

Pursuant to 35 U.S.C. § 119 (e), this application claims priority to the filing date of the U.S. Provisional Patent Application Ser. No. 60/745,570 filed Apr. 25, 2006; the disclosure of which application is herein incorporated by reference.

### BACKGROUND

Topical preparations are used for a variety of different medical purposes. For example, topical preparations known as Band-Aid®, Tegaderm™, Aqua Protect™, Nexcare™, Silverion®, Curad®, Masune®, Coverlet® and Spenco® promote wound healing by protecting cuts and bruises. Other topical preparations are used for transdermal drug delivery in which an active agent is delivered through the skin and into the bloodstream. Exemplary active agents delivered transdermally include scopolamine (for motion sickness), nicotine (for quitting smoking), estrogen (for menopause and to prevent osteoporosis after menopause), and nitroglycerin (for angina). Other topical medical products are applied on the skin but act locally under the topical preparation in the skin, underlying nerves or soft tissues, without any clinically meaningful amount of active medication being absorbed into the bloodstream; examples of this type of topical medication include, Lidoderm™ (lidocaine to relieve the pain of shingles, herpes zoster, topical NSAID preparations, topical menthol preparations, and topical capsaicin products. Other topical products include topical cosmetics.

### SUMMARY

Topical preparation dispensers and methods of using the same are provided. Aspects of the invention include a topical preparation dispenser having a holder, one or more vertically stacked topical preparations in the holder, and a cap at one end of the holder configured such that upon movement of the cap, a topical preparation is exposed and moved relative to the holder. Also provided are kits that include the subject dispensers.

### BRIEF DESCRIPTIONS OF THE DRAWINGS

The figures shown herein are not necessarily drawn to scale, with some components and features being exaggerated for clarity,

FIG. 1 illustrates an embodiment of a cap of a topical preparation dispenser according to the present invention.

FIG. 2 illustrates an embodiment of a topical preparation dispenser according to the present invention.

### DETAILED DESCRIPTION

Topical preparation dispensers and methods of using the same are provided. Aspects of the invention include a topical preparation dispenser having a holder, one or more vertically stacked topical preparations in the holder, and a cap at one end of the holder configured such that upon movement of the cap, a topical preparation is exposed and moved relative to the holder. Also provided are kits that include the subject dispensers.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

As summarized above, the subject invention provides topical preparation dispensers and methods of using the same. In further describing aspects of the invention, components of the subject topical dispensers are reviewed first in greater detail, followed by a review of other aspects of the invention, e.g., methods of using the topical preparations and kits that include the subject dispensers.

### Topical Preparation Dispenser

Aspects of the invention include topical preparation dispensers which include a holder, one or more topical preparations vertically stacked within the holder and a cap at one end of the holder configured such that, upon movement of the cap, a topical preparation is exposed and moved relative to the holder.

The holder of the subject invention is configured to contain one or more vertically stacked topical preparations. In some aspects, the holder includes at least two side walls, a front wall, and a rear wall for holding the vertically stacked topical preparations. In certain aspects, the height of the holder is greater than the width such that the holder is an elongated structure having a height that is greater than the width by 2 or more fold, such as 3 or more fold including 10 or more fold.

The holder may have any configuration suitable for holding vertically stacked topical preparations. Examples of various configurations include, but are not limited to, rectangular, cylindrical, circular, triangular, square or prismatic shaped configurations. In some embodiments, the holder is the same configuration as the topical preparations stacked within its walls. For example, the holder may be rectangular to hold rectangular transdermal topical preparations or circular to hold circular adhesive bandages.

In certain embodiments, the holder has a length that ranges from about 1 cm to about 50 cm, such as from about 2 cm to about 30 cm, and including from about 2 cm to about 20 cm; a width ranging from 1 cm to about 50 cm, such as from about 2 cm to about 30 cm, and including from about 2 cm to about 15 cm; and a height ranging from 1 cm to about 50 cm, such as from about 5 cm to about 30 cm, and including from about 5 cm to about 10 cm.

Certain embodiments include an opening in the front wall of the holder which provides access to the holder for loading topical preparations. In some embodiments, the opening runs the entire length of the holder such that the holder no longer has a front wall, thereby forming a U-shaped cross-section. In other embodiments, the opening is only a portion of the height of the holder such that the opening is less than the height of the holder by 10% or more, such as 25% or more including 50% or more fold.

The subject holder of the present invention includes an end plate positioned at the bottom of the holder. As such the topical preparations are stacked on top of the end plate within the holder. In certain aspects, the area of the end plate is equivalent to the area of the topical preparations. In other aspects, the area of the end plate ranges from about 2 cm² to about 500 cm² or more, e.g., in certain embodiments the area of the end plate ranges from about 4 cm² to about 300 cm², e.g., from about 6 cm² to about 200 cm².

In some embodiments, the holder includes a base for supporting the holder in an upright position. In certain aspects, the base is a separate component positioned at the bottom of the holder. In other aspects, the base is connected to the end plate of the holder as a single component. In some aspects, the base has a length that ranges from 1 cm to about 50 cm, such as from about 2 cm to about 30 cm, and including from about 2 cm to about 20 cm; a width ranging from 1 cm to about 50 cm, such as from about 2 cm to about 30 cm, and including from about 2 cm to about 15 cm; and a height ranging from 1 cm to about 50 cm, such as from about 5 cm to about 30 cm, and including from about 5 cm to about 10 cm.

The holder may be made of any suitable material. In certain embodiments, the holder is made of a material that prevents loss of volatile components and prevents degradation or contamination. Of interest in certain embodiments are materials that protect the topical preparations present therein from environmental factors, such as humidity, microbes, as well as loss of adhesiveness or inactivation of an active agent. Examples of suitable materials include, but are not limited to, polyetherimide; polyethylene, polypropylene, polycarbonate, polymethyl-methacrylate, polyamides, styrene-acrylonitrile polymers, acrylonitrile-styrene gradient polymers and polyesters.

In some embodiments of the invention, the holder includes a desiccant for absorbing moisture. The desiccant may be disposed anywhere within the holder, for example the desiccant may be a coating on the end plate or packed within one of the walls of the holder. The desiccant material may be any suitable material for absorbing moisture, such as but not limited to, zeolites, barium oxide, activated alumina, silica gel, glycerine, magnesium perchlorate, calcium sulfate, calcium oxide, activated carbon, calcium chloride, alumina gel, calcium hydride, phosphoric anhydride, phosphoric acid, potassium hydroxide, sodium sulfate and bentonite clay.

In certain embodiments, the holder includes a compression force element. In some aspects, the compression force element is positioned at the bottom of the holder beneath the end plate. In other aspects, the vertically stacked topical preparations are pressed between the compression force element in the bottom of the holder and the closed cap at the top. The compression force element, in some embodiments, provide the linear force required to compress the vertically stacked topical preparations within the confines of the holder and the cap. In certain aspects, the compression force element provides the upward force required to push the topical preparations upward towards the cap, such that the uppermost topical preparation is positioned within the cap. In other aspects, once the uppermost topical preparation has been dispensed, the compression force element pushes the vertically stacked topical preparations upward such that the next uppermost topical preparation is ready to be dispensed. In some embodiments, the compression force element is a spring and in other embodiments, an elastically deformable band.

As summarized above, the holder is configured to hold a plurality of vertically stacked topical preparations. The number of different topical preparations in a given vertical stack in the holder may vary, but in certain embodiments, the height of the stack ranges from about 2 cm to about 50 cm, such as from about 3 cm to about 40 cm including from about 5 cm to about 25 cm.

Further, the topical preparations may assume a variety of shapes, e.g., rectangular, cylindrical, circular, triangular, square or prismatic. The size of the topical preparation may vary, where in many embodiments the size ranges from about 2 cm² to about 2500 cm², e.g., in certain embodiments the size ranges from about 50 cm² to about 1000 cm² e.g., from about 200 cm² to about 500 cm².

Topical preparations that may be present in the dispensers of the present invention include, but are not limited to, those preparations that have a backing layer having an adhesive. The backing layer is flexible and serves as the primary structural element of the topical preparation. Backings for use in topical preparations are made of a flexible, biocompatible material that imitates the elastic properties of skin and conforms to the skin during movement. In some aspects, the backing layer is derived from synthetic polymers like polyolefin oils polyester, polyethylene, polyvinyl dines chloride, and polyurethane or from natural materials such as cotton, wool, etc.

Adhesives are used with topical preparations to adhere to the backing layer and to adhere to the skin surface. In certain embodiments, adhesives can function under a wide range of conditions, such as, high and low humidity, bathing, sweating etc. Examples of suitable adhesives include, but are not limited to, poyisobutylene-based adhesives, silicone-based adhesives, and acrylic-based adhesives. In some aspects, the adhesive composition is based on natural or synthetic rubber; a polyacrylate such as, polydimethylsiloxane; and hydrogels.

The topical preparations of the present invention are each individually packaged. In certain embodiments, the topical preparations are hermetically sealed in order to maintain sanitary conditions and prevent degradation, contamination, loss of ingredients, loss of sensitivity, etc. The material used for packaging topical preparations may be any suitable material which is impervious to environmental factors and allows the topical preparations to remain stable under storage conditions. Examples of materials which may be used for packaging topical preparations include, but are not limited to, aluminum, polymeric layers, or paper layers.

Embodiments of the present invention include topical preparations that are adhesive bandages. By “adhesive bandage” is meant a flexible strip with an adhesive element, e.g., a coating present on a surface thereof, upon which is placed an absorbent pad, suitable for covering wounds. The adhesive bandages can be any shape or size or can be customized to fit irregularly shaped body parts.

In other embodiments, the topical preparations are used for transdermal drug delivery. By “transdermal drug delivery” is meant the delivery of an active agent across the skin and into systemic circulation by applying a topical preparation to the user's skin surface. In certain aspects, the topical preparation includes an active agent included within the adhesive. In other aspects, the active agent is provided in an additional matrix layer which controls the rate of release. Some aspects of the invention include topical preparations that employ a reservoir system in which the active agent is constantly released through an inert polymeric membrane. In other aspects, the topical preparation may have a microreservoir system for transdermally delivering drugs. In this embodiment, the active agent is located within a medicated polymer disc positioned at the center of the adhesive layer. In other embodiments, the topical preparation are used for “pure topical” drug delivery. By “pure topical” drug delivery is meant the delivery of an active medication or nutraceutical or cosmetic agent crosses the skin but does not become absorbed by the systemic circulation in any clinically meaningful amounts.

In certain aspects, the topical preparations are stacked within the holder in a predetermined sequential order. By “predetermined sequential order” is meant that the topical preparations are loaded in the sequence in which each preparation is to be dispensed. In some embodiments, topical preparations that have different doses of an active agent are loaded in the order the subject is to receive the different doses. In other embodiments, the topical preparations stacked within the device all have the exact same dose.

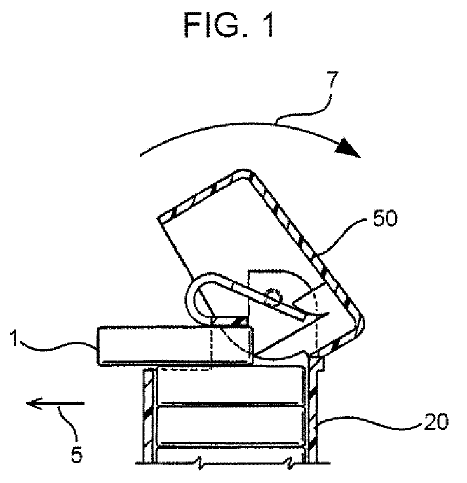

Embodiments of the invention include a cap covering a top of the holder which is configured such that, upon movement of the cap, a topical preparation is exposed and moved relative to the holder. As illustrated in FIG. 1, by “exposed” is meant that the upper most topical preparation (1) is revealed and made accessible to the subject in need of a topical preparation. In certain aspects, the entire topical preparation is exposed to the subject while in other aspects, only part of the topical preparation is revealed. In embodiments where only part of the topical preparation is exposed, the subject in need of a topical preparation grasps the exposed part and pulls the preparation outward in order to obtain a topical preparation. In certain embodiments, 100% of the topical preparation is exposed, while in some embodiments 80%, 60%, 50%, 40% or less is exposed to the subject in need of a topical preparation. By “moving relative to the holder” is meant that the upper most topical preparation (1) moves horizontally away from the holder (in the direction of arrow 5). In certain aspects of the invention, the topical preparation moves from about 1 cm to about 25 cm, such as from about 5 cm to about 20 cm including from about 10 cm to about 15 cm away from the holder.

In certain aspects, the cap (50) is configured to dispense an uppermost topical preparation from the holder (20) upon moving the cap from a closed position to an open position (in the direction of arrow 7). In some embodiments, the cap is connected to the holder by a connector that provides for the movement, as described above. The connector may be a hinge, pin element or any suitable connector known in the art that provides a pivotal movement of the cap. In some embodiments, the cap may be coupled to a compression force element. In certain aspects, the compression force element automatically moves the cap from an open position to a closed position. In certain embodiments, the compression force element is a spring.

In some embodiments, the cap is configured as a decorative head, which has the same function as the cap. In certain aspects, the decorative head is connected to the holder such that, upon movement of the decorative head, a topical preparation is exposed and moved relative to the holder. Examples of decorative heads include, but are not limited to, cartoon characters, historical figures, animals, movie characters, literary figures, holiday characters, and different career figures. Other examples of decorative heads include jewelry, glassware, or beads. Another possible decorative head could include scents, such as perfumes or colognes.

In certain embodiments of the invention, the holder is located within a housing. The housing is configured to provide a protective shell around the holder of the subject invention. As such, in some embodiments, the housing has the same configuration as the holder. However, in other embodiments, the housing may have a completely different configuration from the holder. The housing may be rectangular, cylindrical, circular, triangular, square or prismatic shaped. In some aspects, the housing has a length that ranges from 1 cm to about 1000 cm, such as from about 5 cm to about 500 cm, and including from about 5 cm to about 100 cm; a width ranging from 1 cm to about 1000 cm, such as from about 5 cm to about 500 cm, and including from about 5 cm to about 100 cm; and a height ranging from 1 cm to about 1000 cm, such as from about 5 cm to about 500 cm, and including from about 5 cm to about 100 cm.

The housing may be formed from any appropriate material such as, but not limited to, polystyrene, polyvinyl chloride, polypropylene, polyetherimide, polyethylene, polycarbonate, polymethyl-methacrylate, polyamides, styrene-acrylonitrile polymers, acrylonitrile-styrene gradient polymers and polyesters.

In some aspects, the holder is adapted to be extracted from the top end of the housing for loading the topical preparations. In some embodiments, the housing includes tracks for guiding the holder such that the holder slides along the tracks and is able to extend out of the top of the housing. In some embodiments of the invention, the compression force element is positioned between the bottom of the holder and the housing. In certain aspects, the compression force element provides the force which extends the holder from the housing and provides the force to retract the holder once extended. In certain aspects, 100% of the holder is extended, while in other aspects 80%, 60%, 50%, 40% or less of the holder is extended from the housing.

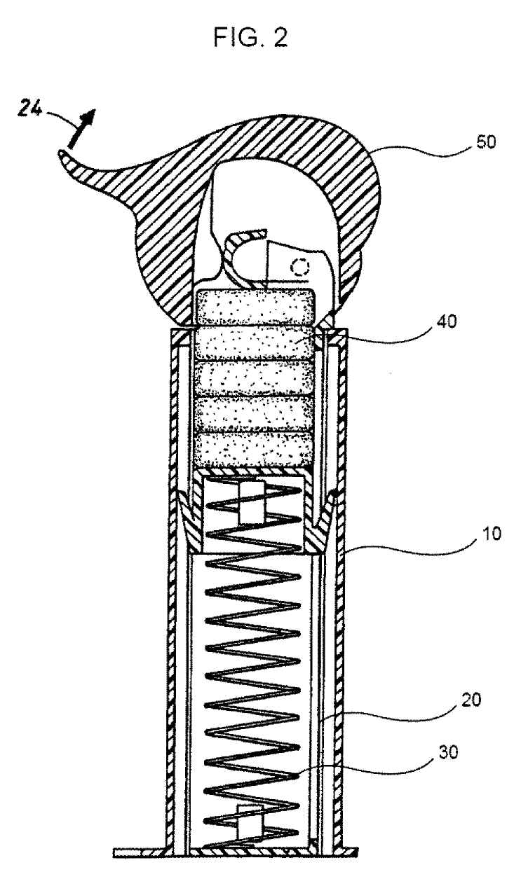

The above subject topical preparation dispensers may be manufactured using any convenient protocol. For example, each component, e.g., the holder, base, endplate, cap, housing and compression force element may be manufactured as a single piece thermoplastic injection molded article. FIG. 2 illustrates an embodiment in which each of the components are assembled to form the subject dispensers. The topical preparation dispenser includes a housing (10) having an opening on top and a holder (20) positioned inside the housing (10). The holder (20) is configured to extend out of the opening on top of the housing (10). A compression force element, e.g., a spring (30) is positioned at the bottom of the holder (20). Within the holder (20), is vertically stacked one or more topical preparations (40). The cap (50) is positioned a top of the chamber to provide a cover and is configured such that, upon movement of the cap in the direction of the arrow, an uppermost topical preparation is exposed and moved relative to the holder.

### Methods

As summarized above, the present invention includes methods of using the subject topical preparation dispensers.

In some embodiments, methods are provided for obtaining a topical preparation. In such embodiments, the cap of the topical preparation dispenser that includes one or more topical preparations is moved in order to expose the uppermost topical preparation contained in the holder. Movement of the cap results in exposing the upper most topical preparation and moving the topical preparation away from the holder. In certain embodiments, the cap is moved from a closed position to an open position in order to expose and move the topical preparation relative to the holder. Once the topical preparation has been exposed and moved, a subject may grasp the exposed part and pull outward in order to obtain a topical preparation.

Other embodiments provide methods for loading the above described topical preparation dispensers. In practicing the subject methods, the cap is moved in order to expose the holder in which vertically stacked topical preparations are then loaded into the holder. In embodiments that include a housing, the cap is pulled upward, thereby extending the holder out from the housing. While the holder is extended, the topical preparations are vertically stacked within the holder. In certain aspects, the topical preparations may be stacked one at a time while in other aspects, an entire stack of topical preparations is loaded at once. In some embodiments, the topical preparations are loaded to fill the entire capacity of the holder. However, in other embodiments, only a specific number of topical preparations are loaded into the holder. For example, only a one month supply of topical preparations is loaded which does not fill the entire capacity of the holder. In other embodiments, the topical preparations are loaded in a predetermined sequential order according to the sequence in which each preparation is to be dispensed.

Some embodiments of the invention include methods for treating a subject having a condition in need of a topical preparation. Generally, “subjects” of the invention are “mammals” or “mammalian,” where these terms are used broadly to describe organisms which are within the class mammalian, including the orders carnivore (e.g., dogs and cats), rodentia (e.g., mice, guinea pigs, and rats), and primates, (e.g., humans, chimpanzees, and monkeys). In many embodiments, the subject is a human suffering from a condition in need of a topical preparation, where the present invention is particularly suited for treating wounds or transdermally delivering an active agent to treat medical ailments, such as to treat aches and pains, cigarette addictions, and motion sickness. Furthermore, the device may be used to house topical cosmetic products.

The first step of the method comprises moving a cap of a topical preparation dispenser to expose a topical preparation present in the holder. The subject then removes the exposed topical preparation and applies the topical preparation to a topical site so that the active agent will be absorbed through the skin in order to treat the condition. The topical preparations of the invention may be applied to any topical area of a subject's body, including but not limited to: arms, legs, face, mouth, neck, back, shoulders, chest, hands, feet, etc.

### Utility

The subject invention may be used in treating a subject having a condition in need of a topical preparation by dispensing a topical preparation which is directly applied to the subject's skin surface.

In some embodiments, adhesive bandages are directly applied over wounds, such as cuts and scrapes in order to prevent infection. In certain aspects, the adhesive bandages may include an antimicrobial agent to prevent infection of the wound. Examples of antimicrobial agents, include but are not limited to, tetracycline, oxytetracycline, chlorotetracycline, neomycin, erythromycin, bacitracin, streptomycin, rifampicin, N-demethylrifampicin, kanamycin, chloromycetin, griseofulvin, mycostatin, miconazole, chlorhexidine, domiphen bromide, domiphen chloride, domiphen fluoride, benzalkonium chloride, dichlorophene, tetrachlorophene, hexachlorophene, zinc pyrithione, silver sulfadiazine, silver uracil and iodine.

In other embodiments, adhesive bandages directly apply pressure to a wound in order to stop the wound from bleeding. In certain aspects, the adhesive bandages include a hemostat to stop the bleeding. Examples of hemostatic agents, include but are not limited to, collagen, thrombin, fibrin, hydrogels, to calcium alginate, glucosamine cationic biopolymers, chitosan, aluminum chloride and ferric salts.

In some embodiments, adhesive bandages are directly applied over wounds, such as cuts and scrapes with active ingredients to minimize associated pain and discomfort, such as itch. In certain aspects, the adhesive bandages may include local anesthetics, such as lidocaine and benzocaine, or NSAID agents, such as ibuprofen, ketoprofen, diclofenac, and others, or opioid drugs, such as morphine, methadone, fentanyl. Furthermore, some embodiments of bandages may include combinations of anti-infectives, hemostatic agents, and pain relieving agents.

In some embodiments, the topical preparations are directly applied to the subject's skin surface for transdermal delivery of an active agent. Transdermal delivery offers controlled release of the active agent into the patient's blood stream and enables a steady blood-level profile, resulting in reduced systemic side effects and improved efficacy.

A transdermal delivery system according to the invention comprises one or more active agents. The active agent, whether it be a medication or nutriceutical agent, included in the topical preparation may be selected from, but is not limited to the following: analgesics, anesthetics, anti-arthritis drugs, anti-inflammatory drugs, anti-headache drugs, cardiovascular active drugs, smoke cessation drugs, hormones, androgen, estrogen, non-steroidal anti-inflammatory agents, anti-hypertensive agents, anti-depressants, antibiotics, anti-cancer agents, antiemetics, anti-infectants, contraceptives, anti-diabetic agents, steroids, anti-allergy agents, nitroglycerin, nicotine, insulin, and vitamins.

In some embodiments of the invention, the topical preparations include active agents in a controlled release formulation. In other embodiments, the topical preparations each have varying amounts of the active agent while in other embodiments, each have a different active agent.

In some embodiments of the invention, the topical preparation include active ingredients, whether it be a medication or nutriceutical agent, that act locally in the skin, nerves, or soft tissues underlying the patch without any resultant meaningful systemic blood levels. For these topical preparations, the active agent may treat, but is not limited to the following: analgesics, anesthetics, anti-arthritis drugs, anti-inflammatory drugs, anti-headache drugs, antibiotics, anti-cancer agents, anti-infectants, steroids, anti-allergy agents, and vitamins.

Another embodiment of this invention includes topical cosmetic preparations.

### Kits

Also provided are kits for practicing the subject methods. The subject kits at least include a topical preparation dispenser for use in practicing the subject methods. The topical preparations may be any type of topical preparation, e.g., adhesive bandages or transdermal patches or topical cosmetic preparations, as described above. In certain embodiments, a kit may include topical preparations which are adhesive bandages that include any of the hemostatic or antimicrobial agents provided above. In other embodiments, the topical preparations include at least one of the above described active agents. Some or all components of the subject kits may be packaged in suitable packaging to maintain sterility.

The subject kits may include instructions for how to load and dispense topical preparations according to the subject methods and how to treat a subject having a condition in need of a topical preparation using the subject topical preparation dispenser. The instructions may be recorded on a suitable recording medium or substrate. For example, the instructions may be printed on a substrate, such as paper or plastic, etc. As such, the instructions may be present in the kits as a package insert, in the labeling of the container of the kit or components thereof (i.e., associated with the packaging or sub-packaging) etc. In other embodiments, the instructions are present as an electronic storage data file present on a suitable computer readable storage medium, e.g. CD-ROM, diskette, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, this means for obtaining the instructions is recorded on a suitable substrate.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

What is claimed is:

1. A topical preparation dispenser comprising:
a holder;
one or more vertically stacked topical preparations in said holder; and
a cap at one end of said holder that is configured such that, upon movement of said cap, a topical preparation of said one or more vertically stacked topical preparations is exposed and moved relative to said holder.

2. The dispenser according to claim 1, wherein said dispenser includes a housing for said holder.

3. The dispenser according to claim 2, wherein said holder is extendable from said housing.

4. The dispenser according to claim 1, wherein said cap includes a compressive force element.

5. The dispenser according to claim 1, wherein said holder includes a compressive force element.

6. The dispenser according to claim 1, wherein movement of said cap is from a closed position to an open position.

7. The dispenser according to claim 1, wherein said holder further includes a desiccant.

8. The dispenser according to claim 1, wherein said topical preparations contain at least one active agent.

9. The dispenser according to claim 8, wherein said active agent is transdermally delivered with systemic activity selected from the group consisting of: analgesics, anesthetics, anti-arthritis drugs, anti-inflammatory drugs, anti-headache drugs, cardiovascular active drugs, smoke cessation drugs, hormones, androgen, estrogen, non-steroidal anti-inflammatory agents, anti-hypertensive agents, anti-depressants, antibiotics, anti-cancer agents, antiemetics, anti-infectants, contraceptives, anti-diabetic agents, steroids, anti-allergy agents, nitroglycerin, nicotine, insulin, and vitamins.

10. The dispenser according to claim 8, wherein said active agent's activity is locally under the application site selected from the group consisting of: analgesics, anesthetics, anti-arthritis drugs, anti-inflammatory drugs, anti-headache drugs, antibiotics, anti-cancer agents, anti-infectants, steroids, anti-allergy agents, and vitamins.

11. The dispenser according to claim 8, wherein said topical preparation includes cosmetic preparations.

12. The dispenser according to claim 8, wherein said at least one active agent is a controlled release formulation.

13. The dispenser according to claim 8, wherein said topical preparations are loaded in said holder in a predetermined sequential order.

14. The dispenser according to claim 8, wherein said topical preparations within said holder each have a varying amount of an active agent.

15. The dispenser according to claim 8, wherein said topical preparations within said holder each have a different active agent.

16. The dispenser according to claim 1, wherein said topical preparations are adhesive bandages.

17. The dispenser according to claim 16, wherein said adhesive bandages include an antimicrobial agent.

18. The dispenser according to claim 17, wherein the antimicrobial agent is selected from the group consisting of: tetracycline, oxytetracycline, chiorotetracycline, neomycin, erythromycin, baci-tracin, streptomycin, rifampicin, N-demethylrifampicin, kanamycin, chloromycetin, griseofulvin, mycostatin, miconazole, chlorhexidine, domiphen bromide, domiphen chloride, domiphen fluoride, benzalkonium chloride, dichlorophene, tetrachlorophene, hexachlorophene, zinc pyrithione, silver sulfadiazine, silver uracil and iodine.

19. The dispenser according to claim 16, wherein said adhesive bandages include a hemostatic agent.

20. The dispenser according to claim 19, wherein the hemostatic agent is selected from the group consisting of: collagen, thrombin, fibrin, hydrogels, calcium alginate, glucosamine cationic biopolymers, chitosan, aluminum chloride and ferric salts.

21. The dispenser according to claim 16, wherein said bandages include pain relieving agents

22. The dispenser according to claim 21, wherein the relieving drug is selected from the group consisting of: local anesthetics, NSAIDs, and opioids.

23. A method for obtaining a topical preparation, said method comprising;
moving a cap of a topical preparation dispenser according to claim 1 to expose a topical preparation present said holder; and
receiving said exposed topical preparation from said holder.

24. A kit for dispensing topical preparations, the kit comprising:
a dispenser of claim 1; and
a plurality of topical preparations.

25. The kit according to claim 24, further comprising instructions for dispensing said topical preparations from said dispenser.

26. A method of loading a topical preparation dispenser comprising a holder and a cap, said method comprising:
opening cap; and
vertically stacking topical preparations in said holder.

27. The method according to claim 26, wherein the dispenser includes a housing.

28. The method according to claim 27, further comprising extending the holder from said housing prior to said vertically stacking topical preparations in said holder.

29. The method according to claim 26, wherein the topical preparations are loaded in said holder in a predetermined sequential order.

30. A method for treating a subject having a condition in need of a topical preparation, the method comprising:
moving a cap of a topical preparation dispenser according to claim 1 to expose a topical preparation present in said holder;
removing said exposed topical preparation present in said holder; and
applying said topical preparation to a topical site of said subject in order to treat said condition.
